(19) United States
(12) Patent Application Publication
(10) Pub. No.: US 2006/0149258 A1
(43) Pub. Date: Jul. 6, 2006

(54) Surgical tool and method for fixation of ligaments

(76) Inventor: Joaquim Paulo Grava de Sousa, Sao Paulo (BR)

Correspondence Address: GABLE & GOTWALS, 100 WEST FIFTH STREET, 10TH FLOOR, TULSA, OK, 74103, US

(21) Appl. No.: 11/011,509

(22) Filed: Dec. 14, 2004

(51) Int. Cl. A61B 17/58

(52) U.S. Cl. 606/72

(57) ABSTRACT

A surgical tool and method for fixation of ligaments employs a wedging element for holding the ligament in contact with supportive tissue (such as bone) of the patient. The wedging element includes an expandable portion that is expanded by operation of an expander element acting upon the wedging element. An internally threaded through opening formed in the wedging element is configured to receive the externally threaded expander element. When the external threading of the expander element is engaged with the internal threading of the wedging element, the expander element is turned relative to the wedging element to drive the expander element into the through opening and thereby expand the expandable portion of the wedging element. In an exemplary use, the wedging element is positioned adjacent a reconstructed ligament and nearby supportive tissue and then expanded to wedge the ligament between the wedging element and supportive tissue, thereby immobilizing the ligament and holding it in contact with the supportive tissue as the ligament heals.

### FIELD OF THE INVENTION

The present invention relates generally to ligament reconstruction. More particularly, the present invention relates to a surgical tool and associate method employing an expansive element for immobilizing reconstructed ligaments such as the anterior cruciate ligament.

### BACKGROUND OF THE INVENTION

The practice of sports and other forms of physical activity is generally seen as an important aspect of a healthy lifestyle. Unfortunately, an active physical lifestyle is usually accompanied by the risk of injury. Rupture of the anterior cruciate ligament (ACL), for example, is today one of the most common knee injuries afflicting orthopedic and emergency room patients. ACL injuries are common among youth while engaged in sports such as soccer where physical exertion and violent impact can rupture the ACL. The rupture mechanism is usually torsional and accompanied by pain and joint effusion in the majority of cases. Diagnosis is usually obtained through clinical examination, x-ray, and/or magnetic resonance imaging. Despite its smallness, the importance of the ACL transcends its dimensions because it is responsible for all anterior stability and equilibrium of the knee.

As is known, especially by experts in the field, injury of the ACL is not easily diagnosed because it is initially accompanied by swelling of the region followed by improvement and gradual reduction of pain. This causes the injury to become more problematic because the patient rarely feels pain during normal daily activities. However, a new sprain is likely to occur during the first excessive physical effort requiring use of the ligament, and the patient then notices that something is wrong. If diagnostic repair of the ACL is not performed in time, there can be deterioration of the menisci and cartilage, rendering the knee immobile.

To address this problem, physicians and surgeons in the relevant field of practice have developed surgical reconstructive treatment procedures aimed at returning the patient to pre-injury performance levels. Autographs are the most commonly used technique and typically involve use of the middle third of the patellar tendon, tendons of the semitendinosus and gracilis muscles, or the quadricipital tendon. ACL reconstruction using the patellar tendon, for example, is a commonly used technique that enables quick recovery by the patient. ACL reconstruction that uses the flexor tendons generally allows for a more tranquil and less painful rehabilitation while avoiding difficulties associated with use of the patellar tendon technique. Such surgical reconstruction techniques use double semitendinosus and gracilis tendons, with femoral fixation employing a femoral metallic transfixing pin and tibial fixation employing a bioabsorbable/metallic interference screw and screws with transfixing washers in the tibia, which enable fixation of the tendons used to replace the ACL.

Although currently known techniques for ACL surgical reconstruction are usually effective, these techniques can also produce undesirable results. For example, the metallic or absorbable interference screws can crush, chew or partially rupture the tendon due to the cutting/abrasive nature of the external threads. Thus, this technique exposes the patient to the risk of rupture, tearing or slackening of the graft. The transfixing screws with indented washers in the tibia can lead to post-operational complications with sinking of the indented washer into the bone, rupturing of the graft during its fixation and slackening of the tendon and posterior arteriovenous injuries, since the screws are fixed without direct view of the posterior region.

What is needed, therefore, is an improved surgical tool and an associate surgical technique for immobilizing/fixating reconstructed ligaments.

### BRIEF SUMMARY OF THE INVENTION

The present invention achieves its objections by providing a surgical tool for use in holding a ligament (such as an anterior cruciate ligament) in contact with supportive tissue (such as bone) of a patient. The surgical tool includes a wedging element having an expandable portion for being positioned adjacent the ligament and supportive tissue. An expander element is used to expand the wedging element so as to wedge the ligament between the wedging element and supportive tissue, thereby inhibiting movement of the ligament as it heals.

In one embodiment, the wedging element includes first and second opposed ends with the first end embodying the expandable portion. A through opening extends from the first end to the second end. The expander element includes an insertion end for being first inserted into the through opening adjacent the second end of the wedging element. A driving end of the expander element operates to drive the insertion end into the through opening to thereby expand the expandable portion of the wedging element.

Interoperability of the wedging and expander elements may be accomplished in a number of ways. In a preferred embodiment, the expander element includes external threading that engages internal threading formed in the through opening of the wedging element. When the external threading of the expander element is engaged with the internal threading of the wedging element, the second end of the expander element is turned relative to the wedging element to drive the expander element into the through opening, thereby expanding the expandable portion of the wedging element. In this embodiment, expansion of the wedging element is controlled by the distance in which the expander element is driven into the through opening.

Ribs may be formed along the external surface of the wedging element as desired or needed to further inhibit movement of the ligament during healing.

The present invention also provides a method for holding a ligament in contact with supportive tissue of a patient. The method includes providing wedging and expander elements substantially as described above, positioning the wedging element adjacent the ligament and supportive tissue, and expanding the wedging element with the expander element so as to wedge the ligament between the wedging element and supportive tissue, thereby inhibiting movement of the ligament as it heals.

### BRIEF DESCRIPTION OF THE DRAWINGS

Preferred embodiments of the invention will now be described in further detail. Other features, aspects, and advantages of the present invention will become better understood with regard to the following detailed description, appended claims, and accompanying drawings (which are not to scale) where:

FIG. 1 is an exploded view of a surgical tool having a wedging element and associate expander element for use in holding a ligament in contact with supportive tissue of a patient according to the invention;

FIG. 2 is an exploded perspective view of the surgical tool of FIG. 1;

FIG. 3 is a partial cross-sectional view of the expander element of FIG. 1;

FIG. 4 is a cross-sectional view of the wedging element of FIG. 1;

FIG. 5 is a cross-sectional view of the surgical tool of FIG. 1 in its unexpanded state;

FIG. 6 is a cross-sectional view of the surgical tool of FIG. 1 in an expanded state;

FIG. 7 is an illustration of the extraction of a portion of the tibia tendon for use in reconstruction of the anterior cruciate ligament according to the invention;

FIG. 8 is an illustration of an exemplary application of the surgical tool of FIG. 1 for reconstruction of a damaged anterior cruciate ligament showing insertion of the semitendinous and gracilis double tendons, with femoral fixation of the reconstructed ligament accomplished by means of a femoral transfixing pin at one end and the wedging element of FIG. 1 positioned for expansion by the expander element so as to achieve tibial fixation of the reconstructed ligament passing through the tibial orifice at the other end;

FIG. 9 is an illustration of the reconstructed anterior cruciate ligament of FIG. 8 with the surgical tool expanded for tibial fixation; and

FIG. 10 is a partial cross-sectional view of the reconstructed anterior cruciate ligament of FIG. 9.

### DETAILED DESCRIPTION OF THE PREFERRED EMBODIMENT(S)

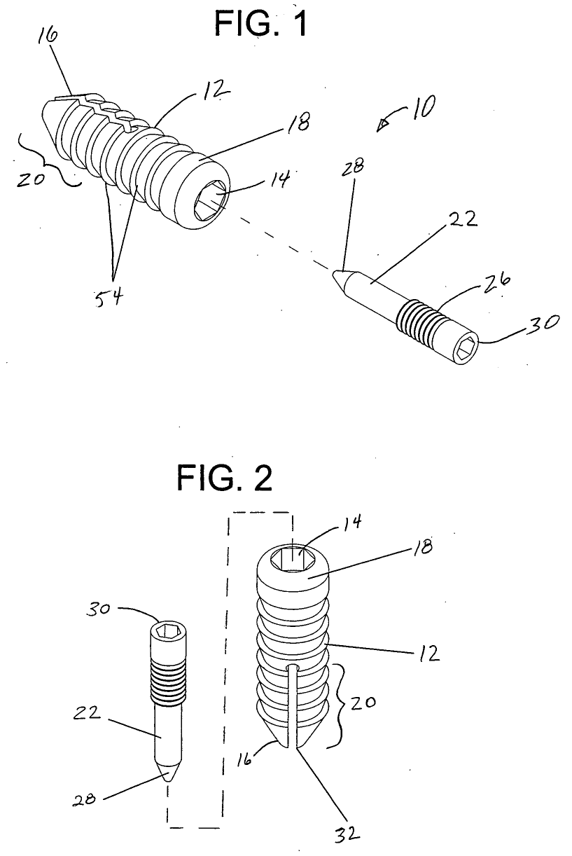

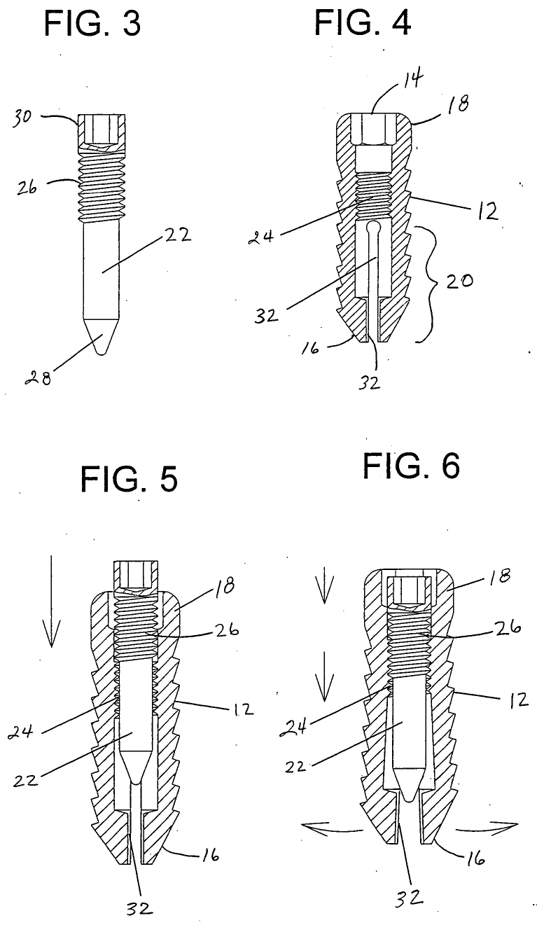

Turning now to the drawings wherein like reference characters indicate like or similar parts throughout, FIGS. 1-4 illustrate a surgical tool 10 for fixation of a ligament to supportive tissue of a patient. The surgical tool 10 includes a wedging element 12, preferably of substantially cylindrical dimension, with a through opening 14 extending from a forward end 16 to a rear end 18. The forward end 16 includes an expandable portion 20 that is expandable by an expander element 22 so as to wedge the reconstructed ligament between the wedging element 12 and supportive tissue (such as bone), as more fully described below.

With continued reference to FIGS. 1-4, the through opening 14 of the wedging element 12 includes internal threading 24 configured to threadingly engage with external threading 26 formed in the expander element 22. In use, an insertion end 28 of the expander element 22 is first inserted into the through opening 14 of the wedging element 12 adjacent rear end 18. When the external threading 26 of the expander element 22 is engaged with the internal threading 24 of the wedging element 12 as shown in FIG. 5, the expander element 22 is turned relative to the wedging element 12 to drive insertion end 28 into the through opening 14 as shown in FIG. 6. To assist in driving expander element 22 into through opening 14, a driving end 30 of the expander element 22 may be configured to receive a driving tool, such as an alien wrench. Expansion of the wedging element 12 is controlled by the distance in which the expander element 22 is driven into the through opening 14.

A slot 32 or other geometrically suitable void formed in expandable portion 20 enables expansion of the outer circumference of expandable portion 20 when the expander element 22 is driven into the through opening 14. In the preferred embodiment shown in FIGS. 1-4, slot 32 is of smaller dimension than that of the expander element 22 so that as expander element 22 is driven into slot 32, the forward end 16 of wedging element 12 is forced expansively outward as seen in FIG. 6.

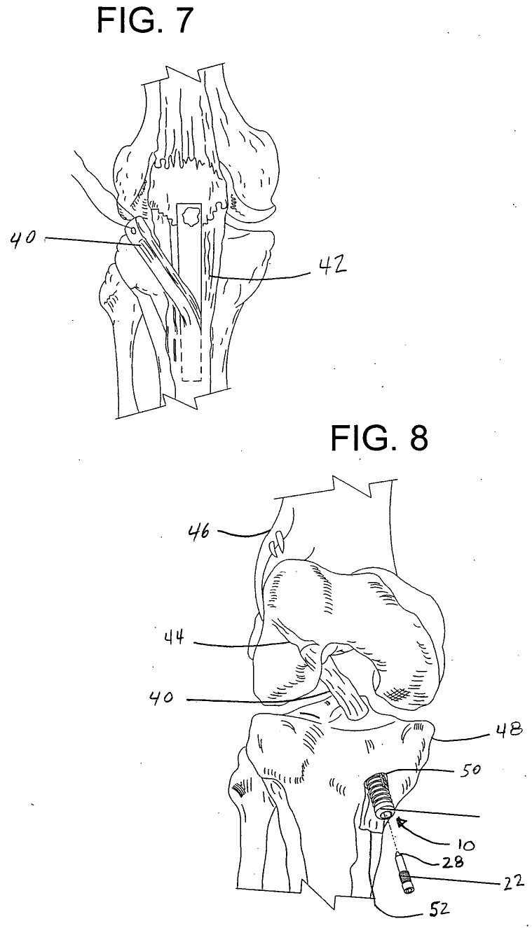

The surgical tool 10 is particularly useful for fixating and immobilizing a reconstructed anterior cruciate ligament (ACL). Reconstruction of the ACL typically involves grafting an extracted portion of a tendon of the patient onto the ruptured ACL. FIG. 7 illustrates extraction of a fragment 40 from the tibia tendon 42. As shown in FIG. 8, the extracted fragment 40 is attached at one end onto the ACL 44 and anchored in the femur 46 by means of a femoral transfixing pin or other suitable fastener. The other end of the reconstructed ACL 44 is held in contact with supportive tissue of the patient by the surgical tool 10, such as the tibia 48, and thereby immobilizes the reconstructed ACL 44 as it heals.

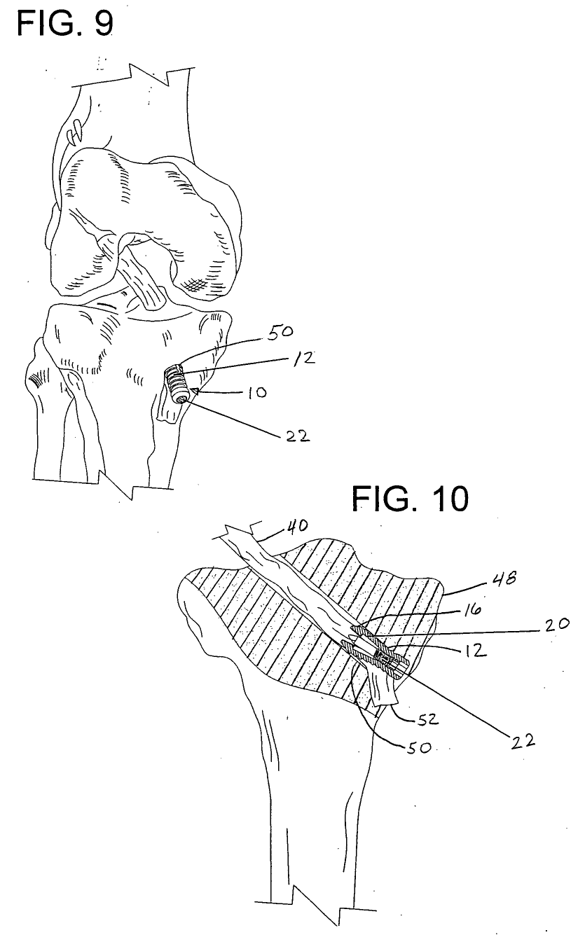

Referring again to the exemplary use of surgical tool 10 shown in FIG. 8, the extracted tendon fragment 40 is fixed to the femur as described above and the extracted tendon fragment 40 of the reconstructed ACL 44 is inserted through the tibial orifice 50. A tapping tool is used to insert forward end 16 of wedging element 12 into the tibial orifice 50 adjacent the free end 52 of reconstructed ACL 44 and supportive tibial orifice 50 as more clearly seen in FIG. 10. Insertion end 28 of expander element 22 is then first inserted into the through opening 14 of expander element 12 and driven into the through opening 14, as described above, so as to expand expandable portion 20 and thereby wedge the reconstructed ACL 44 between wedging element 12 and supportive tibial orifice 50. With the surgical tool 10 fully installed as shown in FIGS. 9 and 10, movement of the reconstructed ACL 44 is inhibited while the ACL 44 heals and fixes itself to the supportive tissue of the tibial orifice 50. Wedging element 12 may be provided with ribs 54 (FIGS. 1 and 9) or other functionally similar structure as desired or needed to further inhibit movement of the ACL 44 during healing.

It will be appreciated that a surgical tool 10 and associate surgical technique according to the invention provide a number of advantages over currently known surgical techniques and devices. For example, the surgical tool 10 employs a wedging technique that significantly reduces the risk of rupturing or tearing of reconstructed ligaments because it does not place the ligament in contact with abrasive structure such as the threading of an interference screw. The wedging action of the surgical tool 10 also provides improved adherence and fixation of the reconstructed ligament in the bone marrow of the tibial orifice 50. The surgical tool 10 is also of relatively simple and inexpensive construction, easy to install, and is highly functional and effective. The surgical tool 10 can also be advantageously employed in a variety of surgical procedures ranging from its use in ACL reconstruction as described herein as well as other surgical interventions that require the fixation of organic components such as ligaments.

The foregoing description details certain preferred embodiments of the present invention and describes the best mode contemplated. It will be appreciated, however, that changes may be made in the details of construction and the configuration of components without departing from the spirit and scope of the disclosure. For example, while the above described wedging and expander elements 12, 22 are shown as separate elements, the two elements may be integrated with each other to accomplish the same function and purpose. Therefore, the description provided herein is to be considered exemplary, rather than limiting, and the true scope of the invention is that defined by the following claims and the full range of equivalency to which each element thereof is entitled.

What is claimed is:

1. A surgical tool for use in holding a ligament in contact with supportive tissue of a patient, the surgical tool comprising:
a wedging element having an expandable portion for being positioned adjacent the ligament and supportive tissue; and
an expander element for expanding the wedging element so as to wedge the ligament between the wedging element and supportive tissue, thereby inhibiting movement of the ligament as it heals.

2. The surgical tool of claim 1 wherein said wedging element is substantially cylindrical.

3. The surgical tool of claim 1 wherein said wedging element is ribbed to further inhibit movement of the ligament during healing.

4. The surgical tool of claim 1 wherein said wedging element further includes:
a first end embodying the expandable portion;
a second end in opposed relation to said first end; and
a through opening extending from the first end to the second end; and
wherein said expander element further includes:
an insertion end for being first inserted into the through opening adjacent the second end of the wedging element; and
a driving end for use in driving the insertion end into the through opening to thereby expand the expandable portion of the wedging element.

5. The surgical tool of claim 4 wherein expansion of the wedging element is controlled by the distance in which the expander element is driven into the through opening.

6. The surgical tool of claim 4 wherein said wedging element includes internal threading and said expander element includes external threading, wherein said expander element threadingly engages the wedging element as the expander element is driven into the through opening.

7. The surgical tool of claim 1 wherein said ligament further comprises an anterior cruciate ligament.

8. The surgical tool of claim 1 wherein said supportive tissue further comprises bone.

9. A surgical tool for use in holding a ligament in contact with supportive tissue of a patient, the surgical tool comprising:
a wedging element having:
an expandable portion for being positioned adjacent the ligament and supportive tissue;
a first end embodying the expandable portion;
a second end in opposed relation to said first end; and
a through opening extending from the first end to the second end; and
an expander element for expanding the wedging element so as to wedge the ligament between the wedging element and supportive tissue, thereby inhibiting movement of the ligament as it heals, said expander element including:
an insertion end for being first inserted into the through opening adjacent the second end of the wedging element; and
a driving end for use in driving the insertion end into the through opening to thereby expand the expandable portion of the wedging element.

10. The surgical tool of claim 9 wherein said wedging element is substantially cylindrical.

11. The surgical tool of claim 9 wherein said wedging element is ribbed to further inhibit movement of the ligament during healing.

12. The surgical tool of claim 9 wherein expansion of the wedging element is controlled by the distance in which the expander element is driven into the through opening.

13. The surgical tool of claim 9 wherein said wedging element includes internal threading and said expander element includes external threading, wherein said expander element threadingly engages the wedging element as the expander element is driven into the through opening.

14. The surgical tool of claim 9 wherein said ligament further comprises an anterior cruciate ligament.

15. The surgical tool of claim 9 wherein said supportive tissue further comprises bone.

16. A method for holding a ligament in contact with supportive tissue of a patient, said method comprising:
providing a wedging element with an expandable portion;
providing an expander element operable to expand the wedging element;
positioning the wedging element adjacent the ligament and supportive tissue; and
expanding the wedging element with the expander element so as to wedge the ligament between the wedging element and supportive tissue, thereby inhibiting movement of the ligament as it heals.

17. The method of claim 16 wherein said step of expanding the wedging element further comprises:
forming an internally threaded through opening in the wedging element;
forming external threading on the expander element;
inserting the expander element into the through opening of wedging element so that the external threading of the expander element engages the internal threading of the through opening of the wedging element; and
turning the expander element relative to the wedging element to drive the expander element into the through opening.

18. The method of claim 16 wherein expansion of the wedging element is controlled by the distance in which the expander element is driven into the through opening.

19. The method of claim 16, further comprising the step of forming ribs along the external surface of the wedging element to further inhibit movement of the ligament during healing.

20. The method of claim 16 wherein said ligament is an anterior cruciate ligament.
